(19) United States
(12) Patent Application Publication
(10) Pub. No.: US 2008/0256688 A1
(43) Pub. Date: Oct. 23, 2008

(54) Article of Eyewear

(75) Inventor: Robert M. Bruce, Portland, OR (US)

Correspondence Address: BANNER & WITCOFF, LTD., 1100 13th STREET, N.W., SUITE 1200, WASHINGTON, DC, 20005-4051, US

(73) Assignee: NIKE, INC., Beaverton, OR (US)

(21) Appl. No.: 11/737,031

(22) Filed: Apr. 18, 2007

(51) Int. Cl. A61F 9/02

(52) U.S. Cl. 2/441

(57) ABSTRACT

An article of eyewear comprises a flexible lens; and a frame having a top, sides, and a bottom for supporting the lens in spaced relation in front of a wearer's face to define an interior space. The frame has a two component surface for mounting the lens thereon. The two component surface comprises a first component and a second component wherein the first component and the second component are configured to collectively provide a tension fit around the face of a user.

### FIELD OF THE INVENTION

The invention relates to an article of sports eyewear having an improved fit for a wearer.

### BACKGROUND OF THE INVENTION

Goggles such as ski goggles, motorcycle goggles, safety goggles and the like provide protection for the wearer's eyes. In particular, the goggles shield the eyes from wind and particulate matter. Some goggles have openings for vents which allow an air flow between the outside and inside of the goggles. The lens may be a single lens or a double lens, and can be tinted for sun and glare protection. The frame of the goggle and lens prevents good peripheral vision and can have distortion. These problems can prevent a good fit against the users face as well as make it difficult to use the goggles within a helmet.

### BRIEF SUMMARY OF THE INVENTION

The present invention is directed to an article of eyewear, in particular to goggles with an improved fit and vision characteristic for the wearer.

In one embodiment, there is provided an article of eyewear comprising a flexible lens; and a frame having a top, sides, and a bottom for supporting the lens in spaced relation in front of a wearer's face to define an interior space. The frame has a composite surface for mounting the lens thereon. The composite surface comprises a first component and a second component wherein the first component and the second component are configured to collectively provide a tension fit around the face of a user.

In one embodiment of an article of eyewear, the first component has a wider portion at the top of the frame. The wider portion can have at least one ventilation aperture. The ventilation aperture can include a movable damper responsive to air flow.

In one embodiment, there is provided an article of eyewear in which a frame has multiple frame members. One frame member can extend from adjacent a first side of the wider portion of the frame member around the periphery to adjacent the wider portion opposite a first side. Another frame member component can include a plurality of air vents.

In one embodiment, there is provided an article of eyewear in which a frame has multiple frame members. A first frame member and a second frame member can have different durometer or stiffness values. In another embodiment, the first frame member has a greater durometer value than the value of the second frame member.

In one embodiment, there is provided an article of eyewear in which the lens is removably attached to the frame. For instance, two apertures are present in the lens, each aperture on an opposite side of the lens and approximate the wearer's temples when eyewear is in use. The lens can be held adjacent the frame via two posts inserted through the apertures and attached to the frame. The posts can be attached to a strap for placing around the wearer's head. Preferably, the posts are pivotally attached to the strap.

In one case, the goggles can be used for sporting activities not limited to skiing, snowboarding, or motorcycling. These and other aspects, features and advantages of the present invention will be readily apparent and fully understood from the following detailed description of preferred embodiments, taken in connection with the appended drawings, which are included by way of example and not by way of limitation with regard to the claimed invention, in which like reference numerals identifying the elements throughout.

### BRIEF DESCRIPTION OF THE DRAWINGS

FIG. 1 is an exploded view of an schematic representation of an article of eyewear in accordance with an embodiment of invention;

FIG. 2 is a perspective view of a schematic representation of an embodiment of a frame used with an article of eyewear;

FIG. 3 is a plan view of an article of eyewear in one in-use position placed around a head of a wearer;

FIG. 4 shows a portion of a strap and post for connection to the lens and frame of the goggles; and

FIG. 5 is a section view of the goggles taken along line 5-5 of FIG. 3.

### DETAILED DESCRIPTION OF THE INVENTIONS

The foregoing summary of the invention, as well as the following detailed description of the preferred embodiments, are included by way of example and not by way of limitation with regard to the claimed invention. The features thereof are described and illustrated in conjunction with materials, constructions, and methods which are meant to be illustrative and non-limiting in scope. For ease of explanation, the article of eyewear is referred to herein as goggles 10.

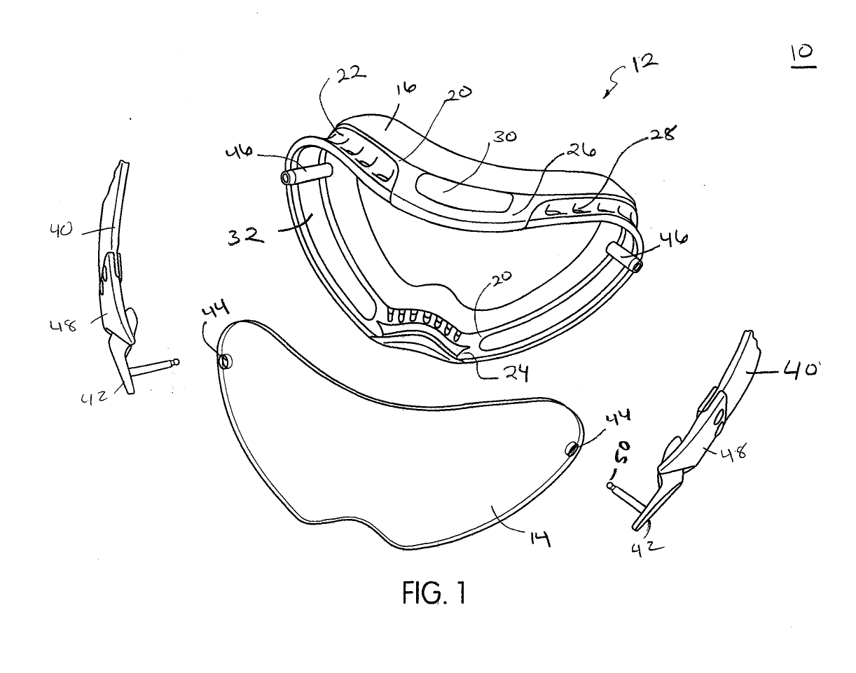

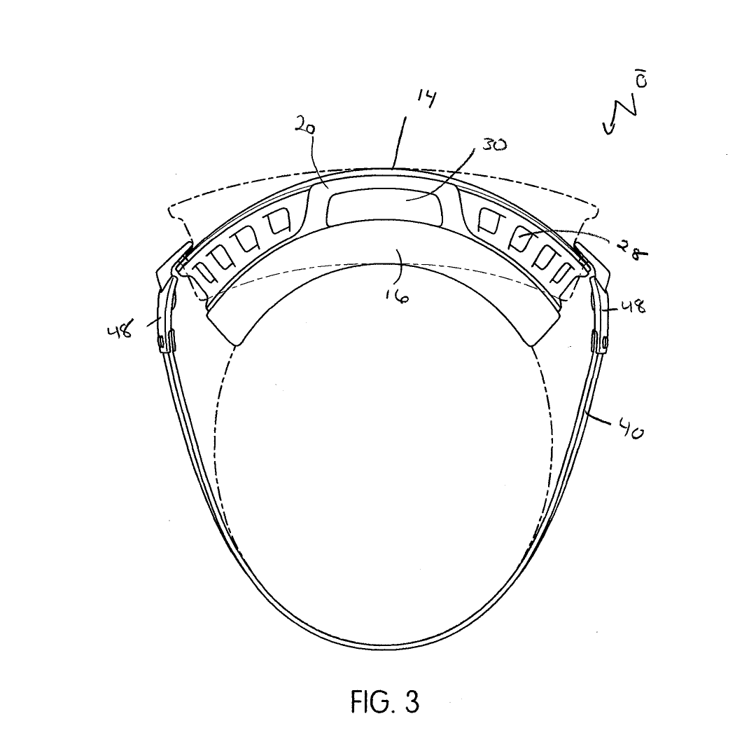

With reference to FIG. 1 and FIG. 3, goggles 10 include a frame 12 which surrounds the eye region of a wearer when in-use. In one embodiment, a lens 14 is removably mounted on the front of the frame 12. The frame 12 and the lens 14 when coupled together define an interior space when the goggles 10 are placed in position against the wearer's head, such as the wearer's face.

The frame 12 is provided with a first frame member 20 and a second frame member 22. These frame members 20, 22 collectively define the frame 12. Nevertheless, other pieces and parts could be provided on the frame. In one embodiment, first frame member 20 is a relatively rigid material that extends around the periphery of the frame 12 and has a wider nose piece section 24 and a wider section 26 at the top central portion of the frame 12. Section 26 contains at least one ventilation aperture 30 having a movable damper responsive to air flow.

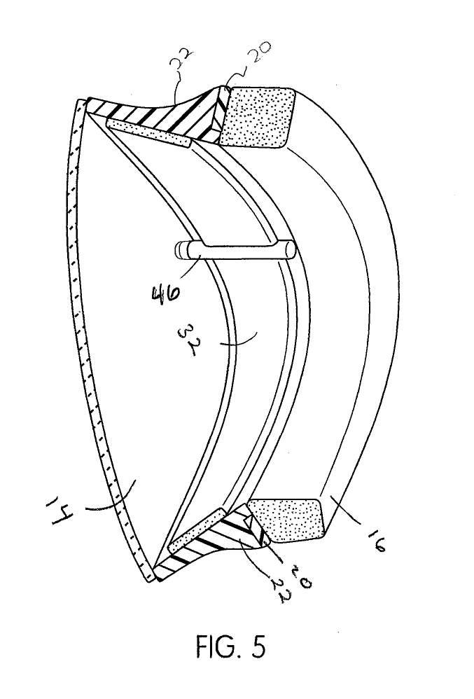

In one embodiment, second frame member 22 comprises a more flexible material than the first frame member 20. As shown in FIGS. 1-3 and 5, second frame member 22 is attached to a narrow band 23 of member 20 and is disposed between first frame member 20 and lens 14. This narrow band 23 of the frame member 20 serves as a base support for the mounting of the second frame member 22 of frame 12. This band of frame member 20 extends around the frame 12. Second frame member 22 extends from adjacent section 26 around the periphery of the lens 14, behind section 24 to adjacent section 26 on the opposite side.

As can be seen in FIGS. 1-3, the second frame member 22 may contain at least two air vents 28 to enable the flow of air into and out of the interior space defined by the frame 12 and the wearer. Air vents 28 may be spaced along the peripheral length of the second frame member 22. Second frame member 22 is provided with a concaved construction around the peripheral length. This concave construction enables an improved channeling of the air (e.g., a fluid) into the air vents 28. The air vents have an inclined portion 27 so as to convey air into the interior space of the goggles 10.

The first frame member 20 and second frame member 22 may be made in a suitable manner. For example, first frame member 20 may be injection molded as one piece construction. In another construction second frame member 22 may be injection molded as a separate component, then bonded or otherwise mounted to the first frame member 20. In yet another construction, second frame member 22 can be injection molded to the first frame member 20. In a construction, the first frame member 20 can be formed with a semi-flexible plastic material, as such nylon. In one construction, second frame member 22 is provided as a soft, resilient element formed of a thermoplastic elastomer (TPE) and Thermoplastic Polyurethane (TPU). To provide optimum fit comfort as well as vision control benefits, the elastomeric material may have a hardness durometer measurement ranging between A15 to A20 Shore hardness. Nevertheless, the hardness of the elastomer could also range between A13 to A25 Shore hardness. Other materials outside this hardness range could also be used.

In one construction, second frame member 22 has a different hardness or flexibility as compared to the flexibility of the first frame member 20. Generally, the material of the second frame member 22 is more flexible (e.g., softer) than the material forming the first frame member 20. In this manner, the frame 20 may be provided different a bendable fit to complement the particular user's cranial anatomical construction. For example, the distal lateral sides of the frame 20 may have a high degree of flexibility to provide more curvature around the wearer's face. (See FIG. 3) The frame and/or lens can be flex inward to provide a greater degree of peripheral vision to the wearer as shown in FIG. 3 at position P2. It is noted that Position P1 shown in broken lines in FIG. 3 is for reference purposes when the goggles 10 are in an unstressed configuration. In another example, the stiffer forward portion provides increased control advantage for fit and ventilation benefits.

In one construction, the first frame member 20 and second frame member 22 have different modulus of elasticity. This construction creates a composite frame structure that provides for a lightweight goggles 10 with greater flexibility. A composite peripheral surface 54 is provided for mounting the lens thereon. In operation of the composite frame structure, the narrow band of the first frame member 20 is constructed of a material with a higher modulus of elasticity, than the modulus of elasticity of the second frame member 22. When the frame 12 is under bending force, the band of first frame member 20 carries a greater load of stress than the second frame member 22. Under the bending force, the second frame member 22 can simultaneously elongate in curvature while providing support for the lens 14. This construction advantageously improves the fit of the goggles around the cranium as well as improves the peripheral vision of the wearer.

A thin, air-permeable, foam strip 32 may cover a portion of the interior of second frame member 22 including vents 28. The foam strip distributes the air flow from the vents 28 and serves to filter particulate matter. The foam strip can be glued or secured over the inside openings of the vents 28. The foam strip 32 is provided as open cell porous foam. The strip 32 serve s to prevent snow or other particulates from entering through the air vents into the interior of the goggle. A lesser dense foam will admit more air flow into the goggle interior. A dense foam will admit less air and can be selected to block most air from entering the goggle interior if desired. The thickness and density of the foam is selected to control the air flow and disburse it in a more even flow throughout the interior of the goggle space. The foam strip may be of any suitable thickness and is typically 2-4 mm thick.

Generally, air flow may occur during skiing or when moving on a motorcycle, for example. Typically, during movement, the air flows by the upper and side vents and through vent aperture 30, and out through the lower and side vents depending upon the positioning of the side vents 28. The air flow patterns can be in a generally downward direction through the interior space and partly against the inside of the front lens. It is further possible for the air to flow to create somewhat a slight vacuum effect to pull through the interior of the goggle structure between the face and lens.

In one construction, the vents 28 are located in the bottom section of the frame 12 so as to draw air from the top of the frame downwardly in order to exit at the bottom of the frame in the vicinity of the nose region. This serves to disperse moisture-laden air from the wearer's nose and reducing fogging within the goggle.

The thin foam strip 32 covering the vents 28 at the top of the frame 12 may be different from the foam which covers the vents at the bottom. For example, the foam at the top may be thicker and have a different porosity than the foam at the bottom.

The frame 12 may include at least one flexible padding 16 attached to first frame member 20 to provide a comfortable fit against the user's face. The padding provides comfort to the wearer as well as provides a snug fit against the face to avoid particulates from passing between the frame and the face.

A strap 40 is attached to the frame 12 to secure the goggles 10 to the user's head or to the back of a helmet. The strap 40 may include a buckle adjustment (not shown) in order to adjust its length so as to fit the goggle snugly against the users face. Such straps are well known in the art and can be of various thicknesses. The strap may be formed of any suitable material such as conventional elastic woven nylon material.

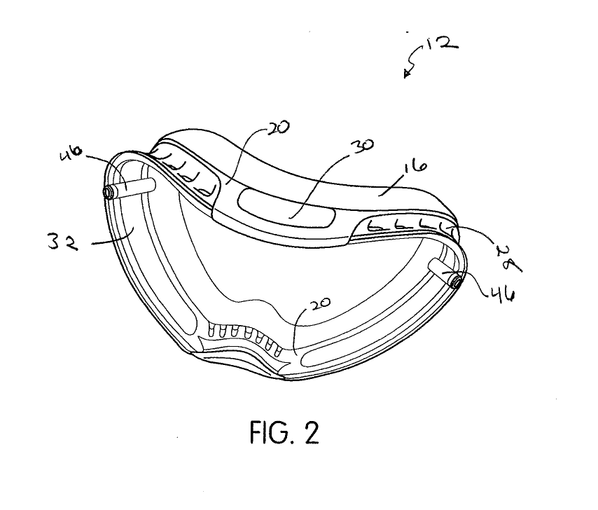

When viewing FIG. 2, Posts 42 are pivotally attached to strap 40 through strap holding connectors 48. The post may be any suitable shape and may be made of any suitable rigid material such as a hard plastic or metal. The post has a head portion 50 that holds the lens against the frame. In one construction, the post has an elongated flat top, wherein one end has the post and the other end is attached to strap holding connector 48.

The strap holding connectors 48 may be attached to the strap by any suitable means such as rivets. The strap holding connectors 48 may be any suitable material such as plastic or metal. The posts 42 are pivotally connected to the strap holding connectors 48 via a pin or a snap connection.

FIG. 1 illustrates an exploded assembly view of the goggles 10. Lens 14 has two apertures 44, each aperture is disposed on an opposite side of the lens near its periphery about the position of the temples of a wearer, if the goggles were in an in-use position. The frame 12 has two elongated receiver members 46 to matingly receive the posts 42 therein and correspond to the same positions as the apertures 44 in the lens 14. The lens 14 is placed against the frame 12 with the aperture of the lens 44 aligning with respective receivers 46. The posts 42 of strap holding connectors 48 pass through the apertures 44 and into the interior void space of receiver members 46 to reliably mount the lens 14. The receiver members 46 can be a suitable shape. In one construction, shown in FIGS. 1-2 and 5, the receiver members 46 are of a cylindrical shape and are part the of the first frame member 20. Hence, receiver members 46 can be formed by injection molding with the first frame member 20.

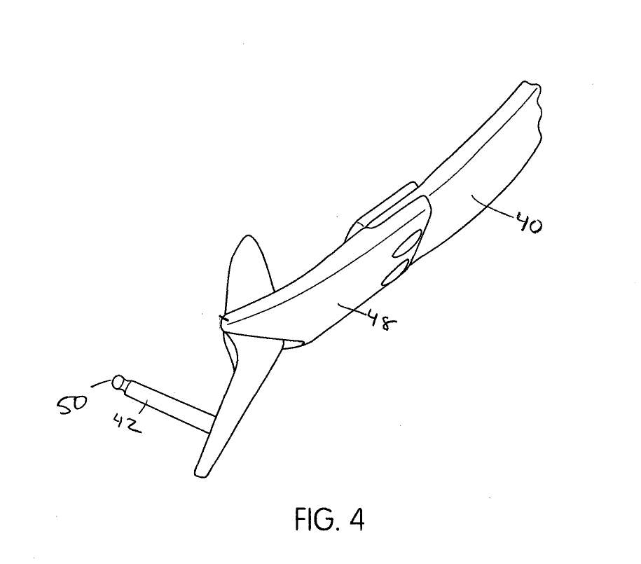

The posts are locked into place by any suitable means. For example, as shown in FIGS. 1 and 4, each post 42 may contain a ball 50 at its distal end which is received by a socket (not shown) in each receiver member 46. Generally, the ball 50 may be pressed into the socket with pressure, but may also be pulled out from the receiver if the lens is changed. Then the strap can be placed over a wearer's head or over a helmet, and can be snugly adjusted to be retained by the wearer's head or helmet. Although round apertures and receivers are illustrated, any suitable shape may be used depending on the shape of the post. During assembly, the strap attached to the frame by inserting the post 42 through the aperture in both sides of the lens and into the receivers. Pressure is applied to the post to lock the post into place. The post is secured so as to not pull out when retention forces are placed on the strap.

The plastic lens 14 can have a narrow gasket attached to the periphery of the lens to provide a sealed connection to the frame. The lens 14 can be a single lens or a double lens for thermal protection, as is conventional. In addition, the lens 14 can be tinted with various colors to affect contrast characteristics.

In one embodiment, the goggles 10 includes a flexible frame allowing a larger frame to fit around a wearer's head. A larger frame allows a larger lens which in turn provides good peripheral vision and depth perception to the wearer of the goggles. Good peripheral vision and depth perception provides a better sense of balance during the desired sport. In another embodiment, goggles 10 may include interchangeable lenses that are tension fit against the frame. The lens may be a single or double lens, clear or tinted. In another embodiment, goggles 10 include an interchangeable strap. The strap is attached to the frame using posts which also hold the lens in place on the frame. In another embodiment, goggles 10 may have vents to allow airflow into and out of the space created between the lens frame and the wearer's face. At least one vent is a ventilation aperture having a movable damper. The damper allows a flow of air into the space during activity.

In operation, the previously described features, individually and/or in any combination, improve the fit and vision performance for article eyewear. Other constructions of an article of eyewear are possible. While the various features of the goggles 10 work together to achieve the advantages previously described, it is recognized that individual features and sub-combinations of these features can be used to obtain some of the aforementioned advantages without the necessity to adopt all of these features in an article of eyewear.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

I claim:

1. An article of eyewear comprising:
a lens; and
a frame for supporting the lens in spaced relation from a face of a wearer,
the frame having a composite peripheral surface for mounting the lens thereon; the composite surface comprising a first component and a second component being mounted to the first component, wherein the first component and the second component are configured with different flexibility to collectively provide a tension fit around the face of a wearer.

2. The article of eyewear according to claim 1 wherein the different flexibility pertain to modulus.

3. The article of eyewear according to claim 1 wherein the first component has a smaller flexibility value than the flexibility value of the second component.

4. The article of eyewear according to claim 1 wherein the first component comprises a wider portion at the top of the frame, the wider portion comprising at least one ventilation aperture.

5. The article of eyewear according to claim 4 wherein the ventilation aperture includes a movable damper responsive to air flow.

6. The article of eyewear according to claim 1 wherein the second component includes a plurality of air vents.

7. The article of eyewear according to claim 1 wherein the second component extends from adjacent a first side of the wider portion of the first component around the periphery to adjacent the wider portion opposite the first side.

8. The article of eyewear according to claim 7 wherein the lens is adjacent the wider portion of the first component and the second component.

9. The article of eyewear according to claim 1 wherein the first component extends around the periphery of the frame.

10. The article of eyewear according to claim 1 wherein the lens is removably attached to the frame.

11. The article of eyewear according to claim 10 further comprising two apertures in the lens, each aperture on an opposite lateral side of the lens; wherein the lens is abutted against the frame via two posts inserted through the apertures providing removable attachment to the frame.

12. The article of eyewear according to claim 1 further comprising an interchangeable strap.

14. The article of eyewear according to claim 13 wherein the straps comprise the posts for attaching the lens to the frame.

15. The article of eyewear according to claim 14 wherein the posts are pivotally attached to the strap.
